(19) United States
(12) Patent Application Publication
(10) Pub. No.: US 2018/0340213 A1
(43) Pub. Date: Nov. 29, 2018

(54) Multiplex Quantitative Polymerase Chain Reaction In One Reaction

(71) Applicant: DIATHERIX Laboratories, Inc., Huntsville, AL (US)

(72) Inventors: Danielle JOHNSON, Huntsville, AL (US); Cheryl SESLER, Athens, AL (US); Stefan BRZEZINSKI, Decatur, AL (US); Elena GRIGORENKO, Madison, AL (US)

(73) Assignee: DIATHERIX Laboratories, Inc., Huntsville, AL (US)

(21) Appl. No.: 15/605,344

(22) Filed: May 25, 2017

(51) Int. Cl. C12Q 1/68; C12N 9/22

(52) U.S. Cl. CPC C12Q 1/686; C12N 9/22

(57) ABSTRACT

Disclosed are methods for quantitative multiplex PCR in which any combination of target sequences can be paired with each other. The reaction mix for use in these methods can be assembled all at one time and the method performed without additional manipulation between the first and last rounds of replication.

### FIELD OF THE INVENTION

The invention described herein relates to multiplex polymerase chain reaction (PCR) methods, including multiplex methods that can quantify the concentration of individual nucleotide templates in a sample.

### BACKGROUND

So-called “real-time” PCR technologies are useful for measuring concentrations of target nucleotides in a PCR reaction. For this reason, real-time PCR is also frequently designated “quantitative PCR” or “qPCR.” See, e.g., U.S. Pat. No. 7,972,828 to Ward et al.

Multiplex polymerase chain reaction (PCR) allows the amplification of multiple loci, potentially from multiple organisms, in one reaction using multiple sets of locus specific primers. However, multiplex PCR has historically been limited by cross reactivity and mutual inhibition among the various primer pairs. Therefore, for qPCR to be run in multiplex fashion has historically required more careful balancing than end-point multiplex PCR. See, e.g., U.S. Pat. No. 5,876,978 to Willey et al. In addition quantitative multiplex PCR is unable to use the more convenient features of non-multiplex qPCR, such as fluorescence intensity read-outs from SYBR™ dyes.

### SUMMARY

The present disclosure describes kits and methods that are useful for multiplex qPCR. In certain embodiments, the PCR reactions described herein can be monitored in real-time based on gradually increasing fluorescence intensity. In additional embodiments, the PCR reactions provide quantitative data only at the end of the reaction process.

For example, in one embodiment a method is disclosed herein for quantitative multiplex amplification, in which the method comprises: (a) amplifying a plurality of target sequences in a cycler containing a single reaction mix, wherein a pair of target enrichment primers comprising a forward flanking (F_{f}) and a reverse flanking (R_{f}) primer each hybridize to a sequence adjacent to the target sequence and wherein F_{f} and R_{f} both anneal at a first annealing temperature; and (b) amplifying the plurality of target sequences in the single reaction mix, wherein a pair of target amplification primers comprising a forward inner (Fᵢ) and a reverse inner (Rᵢ) primer each hybridize to a portion of the target sequence. Fᵢ and Rᵢ both anneal at a second annealing temperature. The second annealing temperature is at least 3 C.° cooler than the first annealing temperature. The single reaction mix comprises: (i) a thermostable DNA polymerase with 5′ to 3′ exonuclease activity; and (ii) a plurality of sequence-specific probes. There is at least one probe complementary to each target sequence in the plurality, and each sequence-specific probe comprises at least one fluorophore and a quencher. The at least one fluorophore responds to an excitation wavelength by emitting a first fluorescence, and the quencher quenches the first fluorescence prior to hydrolysis of the probe. The cycler is equipped with filters responsive to a plurality of first fluorescence wavelengths, and an illumination source periodically illuminates the single reaction mix for detection.

Additional details and exemplary embodiments are disclosed hereinafter.

### BRIEF DESCRIPTION OF THE DRAWINGS

FIG. 1 shows a flow chart of a multiplex PCR method.

FIG. 2 shows a flow chart of a multiplex PCR method. Although SYBR Green® is shown in FIG. 2 for purpose of illustrative example, the method described is not limited to embodiments with SYBR Green®.

### DETAILED DESCRIPTION

As used herein, all nouns in singular form are intended to convey the plural and all nouns in plural form are intended to convey the singular, except where context clearly indicates otherwise. As used herein, “and/or” includes any and all combinations of one or more of the associated listed items.

In certain embodiments, the methods and compositions described herein may be used to diagnose disease agents. As used herein, an “agent” means any organism, regardless of form, that incorporates a nucleic acid and that causes or contributes to an infection, a symptom, or a condition, including, but not limited to a bacteria, a virus (regardless of RNA or DNA genome), or a eukaryotic parasite. The infection, symptom, or condition is designated a “disease state” herein. As used herein, a “disease agent” is an agent that causes or contributes to a disease state. In certain embodiments the disease agent may be involved in bio-weapons programs, such as the organism described as potential biothreats which are described in the NIAID Biodefense Research Agenda.

The methods and compositions described herein are useful in detecting and/or quantifying one or more “target” sequences. As used herein, a “target” sequence is any sequence whose presence, absence, or concentration in a sample can provide useful information for making a given determination (e.g., “does this patient have a Staphylococcus aureus infection?”, or “is this pool water contaminated with Giardia lamblia?”). In certain embodiments, the methods described herein can be used to detect “at least one target.” In the context of the present specification “at least one target” implies at least two sequences to be detected, viz. the target of interest that conveys information about the study sample (e.g., a patient sample or an environmental sample) and a control sequence whose measurement makes it possible to interpret the results regarding the target sequence(s) of interest.

As used interchangeably in this disclosure, “nucleic acid molecule,” “oligonucleotide,” and “polynucleotide” include RNA or DNA, whether single or double stranded, and regardless of whether coding, complementary, or antisense. “Nucleic acid molecule,” “oligonucleotide,” and “polynucleotide” also include RNA/DNA hybrid sequences in either single chain or duplex form. As used herein, “nucleotide” can be an adjective to describe molecules comprising RNA, DNA, or RNA/DNA hybrid sequences of any length. More precisely, “nucleotide sequence” encompasses the nucleic material itself and is thus not restricted to the sequence information (i.e., the succession of nucleotide bases) that biochemically characterizes a specific DNA or RNA molecule. As used herein, “nucleotide” can also be a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. As used herein, “modified nucleotides” comprise at least one modification such as (a) an alternative linking group, (b) an analogous form of purine, (c) an analogous form of pyrimidine, or (d) an analogous sugar. Polynucleotide sequences described herein may be prepared by any known method, including synthetic, recombinant, ex vivo generation, or a combination thereof, as well as any purification methods known in the art.

The compositions and methods described herein are capable of detecting disease agents that cause or contribute to a variety of disease states. In particular, these compositions and methods can be used in differential diagnosis to determine if a specific disease agent is present and to determine if secondary disease agents are present. However, the compositions and method described herein may also be used to determine the presence or absence of genetic mutations related to disease states, the presence or absence of single nucleotide polymorphisms (SNPs), to determine gene expression profiling, and to determine gene dosage mutations. These compositions and methods can also be used to determine the presence, absence, or concentration of a biologic agent in a given environmental setting, such as a drinking water reservoir, a pond, a lake, a beach, a sewage treatment plant, a feed lot, a food supply, and/or a beverage supply. Applications of these alternative uses are described in US 2004/0086867. Other uses of the compositions and methods described herein will be apparent to those skilled in the art.

All patents and published patent applications referenced herein are incorporated by reference in their entireties. Where definitions conflict as between the present text and texts incorporated by reference, the definitions of the present text control.

### Nucleic Acid Isolation

In certain embodiments, nucleic acids can be isolated prior to detection. In certain embodiments, both RNA and DNA are isolated in a single reaction. In other embodiments, RNA and DNA may be isolated independently. In additional embodiments, the individually isolated DNA and RNA can be combined prior to detection. A variety of techniques and protocols are known in the art for RNA and/or DNA isolation. The nucleic acid isolation techniques may be used to isolate nucleic acid from a variety of patient samples or sources. Such patient samples/sources include, but are not limited to, cerebrospinal fluid, nasal/pharyngeal swabs, saliva, sputum, serum, whole blood, and stool.

In certain embodiments, nucleic acid isolation may inactivate infectious agents in the sample, thus reducing any risk to laboratory and healthcare personnel. In such circumstances, requirements for stringent bio-containment procedures may also be relaxed for the remaining steps of the PCR analysis. In addition, DNA and/or RNA isolation may remove enzymatic inhibitors and other unwanted compounds from the isolated nucleic acid, thus making the subsequent PCR more efficient.

In one embodiment, a dual RNA/DNA isolation method is used employing an affinity resin (e.g., QIAGEN® DNEasy® and/or RNEasy® technologies) for initial isolation of RNA and/or DNA from patient samples. Wash steps may be used to remove PCR and RT-PCR inhibitors. The column method for nucleic acid purification is advantageous as it can be used with different types of patient samples and the spin and wash steps effectively remove PCR or RT-PCR inhibitors.

### Reverse Transcription

Additionally or alternatively, where an RNA genome or an RNA target is present, reverse transcription (“RT”) PCR may be utilized. PCR and RT-PCR methodologies are well known in the art.

### Probe-Based Detection

In certain embodiments, amplification products are detected using fluorescently labeled nucleotide probes. The probes can contain sequences complementary to the amplicon to be detected. In certain embodiments, the probes may also contain non-complementary sequences at one or both ends. The complementary sequence of each probe can be any length (e.g., at least 5 bp, at least 10 bp, at least 15 bp, at least 20 bp, at least 25 bp, at least 30 bp, at least 35 bp, at least 40 bp, at least 45 bp, or at least 50 bp), but the person of ordinary skill will appreciate that the length of the complementary sequence will affect both melting temperature and target specificity. In certain embodiments, the probes can be designed to have an annealing temperature that is materially lower than the F_{f}/R_{f} primers, but substantially similar to the annealing temperature of the Fᵢ/Rᵢ primers. The probes contain a molecule capable of fluorescence at a given excitation wavelength (“fluorophore”) on one end and a molecule capable of quenching the fluorophore's fluorescence (a “quencher”). For example, a given probe might contain OREGON GREEN® on one end and BHQ1® on the other. Additionally or alternatively, a given probe might contain CY3® on one end and BHQ2® on the other. Additionally or alternatively, a given probe might contain CY5® on one end and BHQ3® on the other. Additionally or alternatively, a given probe might contain FAM® on one end and MGB® on the other. Additionally or alternatively, a given probe might contain VIC® on one end and MGB® on the other. Additionally or alternatively, a given probe might contain NED® on one end and MGB® on the other. Additionally or alternatively, a given probe might contain VIC® on one end and QSY® on the other. Additionally or alternatively, a given probe might contain JUN® on one end and QSY® on the other. Additionally or alternatively, a given probe might contain FAM® on one end and QSY® on the other. Additionally or alternatively, a given probe might contain CY5® on one end and Iowa Black RQ® on the other. Different quenchers require different degrees of physical proximity to their respective fluorophores, and the person of ordinary skill will know how to adjust the length of the probe as necessary to ensure that the quencher is properly positioned relative to the fluorophore.

### Real-Time Multiplex PCR

The methods described herein allow for efficient amplification of multiple target sequences without extensive empirical testing of primer combinations and amplification conditions as is required with other multiplex amplification methods known in the art. Detection of an amplified target sequence indicates the presence and identity of the target in the sample of interest, thereby providing useful information. In one embodiment, a single target sequence is selected for amplification from each of a set of disease agents to be tested. In an alternate embodiment, more than one target sequence is selected for amplification from each disease agent in the set.

In the methods described herein, the cycling occurs in two steps. In the first step, various target sequences are amplified with one set of primers in a target enrichment step. After the target enrichment, a second set of primers can amplify using the target-enrichment amplicons as template-target amplification step. In certain embodiments, probes, primers, and/or enzymes can be added before the start of the amplification reaction, and then the reaction can proceed without further input from the operator.

While the methods described herein allow for flexibility and freedom of primer pairing, these methods also allow one to quantify template concentrations by assembling one and only one reaction mix, i.e. a single reaction mix. That is to say, the user can assemble the elements necessary for the multiplex PCR method, cap the tubes, and start the amplification. Although a human user is free to reopen the tubes and adjust the reaction mix if one so desires, the methods described herein make it possible that once the reaction mix is first assembled and the temperature cycling begun, no further human intervention is necessary. The reduced necessity for human intervention makes the methods described herein potentially faster, potentially less expensive, and potentially more amenable to high-throughput processing than other quantitative multiplex PCR methods.

For target enrichment, the user provides a series of target enrichment primers for each of a set of target sequences to be analyzed. Each target is defined by at least two “target enrichment” primers: a forward outer primer, also called a forward flanking (F_{f}) primer; and a reverse outer primer, also called a reverse flanking (R_{f}) primer. Target amplification then proceeds using a forward inner primer (Fᵢ); and a reverse inner primer (Rᵢ). The Fᵢ primer is substantially identical in sequence to the 5′ end of the top strand of the target sequence, while the Rᵢ primer is substantially complementary to the 3′ end of the top strand of the target sequence. The Fᵢ and Rᵢ primers can each be any length greater than 5 nucleotides, for example greater than 10, greater than 15, greater than 20, greater than 25, greater than 30, greater than 35, greater than 40, greater than 45, or even greater than 50 nucleotides in length. In practice, Fᵢ and Rᵢ primers will each typically be no less than 12 nucleotides in length but no more than 40 nucleotides in length. The Fᵢ and Rᵢ primers do not have to be each identical in length to the other, but it is useful that the melting temperatures for each be no more than 5 C.° apart, for example no more than 3 C.°, no more than 2 C.°, or no more than 1 C.° apart. In certain embodiments, the Fᵢ and Rᵢ primers will have identical melting temperatures. Those of ordinary skill know how to calculate the melting temperature of a PCR primer, and will thus understand that the melting temperature is proportional both to the total length of the primer and to the G/C content.

Although various embodiments of the methods and compositions disclosed herein involve multiple Fᵢ/Rᵢ primer sets, in which each Fᵢ/Rᵢ primer set binds to a unique target, various Fᵢ/Rᵢ primer sets can share a common set of tags at the 5′ end of each primer. In certain embodiments, all Fᵢ/Rᵢ primer sets share a common set of tag sequences. In other embodiments, the Fᵢ/Rᵢ primer sets are untagged.

The F_{f} primer is substantially identical in sequence to a sequence adjacent to but upstream of the top strand of the target, while the R_{f} primer is substantially complementary to a sequence adjacent to but downstream of the top strand of the target. As used herein, “adjacent” is not limited to “immediately adjacent.” For example, in the sequence A-B-C-D-E-F-G . . . [etc], A is immediately adjacent to B, but not adjacent to G. However, in certain circumstances, it is appropriate to describe A as being adjacent to C, even though A is not immediately adjacent to C. With more specific reference to nucleotides, to say that the F_{f} primer and/or the R_{f} primer is “adjacent” to the target means that the primers each bind to a sequence near enough to the target that the primers can still prime amplification of the target, even though the location of primer binding may not be immediately adjacent to the target. The F_{f} and R_{f} primers do not have to be each identical in length to the other, but it is useful that the melting temperatures for each be no more than 5 C.° apart from each other, for example no more than 3 C.°, no more than 2 C.°, or no more than 1 C.° apart. In certain embodiments, the F_{f} and R_{f} primers will have identical melting temperatures. In certain embodiments, the lowest melting temperature of the F_{f}/R_{f} primer sets will be at least 3 C.°, for example at least 4 C.°, at least 5 C.°, at least 6 C.°, at least 7 C.°, at least 8 C.°, at least 9 C.°, at least 10 C.°, or even at least 15 C.° greater than the highest melting temperature of the corresponding Fᵢ/Rᵢ primer set. In certain embodiments, the F_{f}/R_{f} primers can be used by themselves—i.e., without Fᵢ/Rᵢ primers—to amplify a target sequence.

The specificity of the hybridization between the target enrichment primers and their nucleic acid sequences can be adjusted by increasing or decreasing the length of the primer sequence responsible for hybridization as is known in the art. In general, a longer primer sequence will give increased specificity. Increasing or decreasing the lengths of the primer sequence responsible for hybridization may also determine which primers are active during the various stages of the amplification process. In one embodiment, the length of the target enrichment primers is from 10 to 50 nucleotides. In one embodiment, the length of the target enrichment primers is from 10 to 40 nucleotides. In one embodiment, the length of the target enrichment primers is from 10 to 20 nucleotides. Each target enrichment primer may be a different length from the others if desired. For example, in one embodiment, the F_{f} and R_{f} are each 35-45 nucleotides in length, while Fᵢ and Rᵢ are each 15-25 nucleotides in length (with such length not including any binding tag sequence).

Any convenient target sequence can be chosen for amplification and detection, so the nucleotide sequences of the target enrichment primers are dictated only by the nature of the nucleic acid sequence flanking the target sequence. Therefore, the target enrichment primers can be designed with minimal constraint on their composition. Multiple sets of target enrichment primers may enhance the sensitivity and specificity of the assay by allowing more opportunity and combinations for nested primers to work together to provide target sequence enrichment. More than two sets of target enrichment primers may be used if desired. In one embodiment, 2 to 6 sets of target enrichment primers are used. In one embodiment, 3 to 5 sets of target enrichment primers are used. In one embodiment, 3 to 4 sets of target enrichment primers are used.

In certain embodiments, target amplification can employ at least one set of“super” primers designated “forward super primer” (FSP) and “reverse super primer” (RSP). The super primers are optional for the methods described herein, but when they are used, they will typically be used in connection with sets of Fᵢ/Rᵢ primers that are tagged on their 5′ ends. The super primers can be complementary to these tag sequences. When they are used, the super primers may be present in the reaction mix during the target enrichment step. However, in the methods disclosed herein, the target enrichment primers can be designed to have a materially higher annealing temperature than the target amplification primers. Because of this difference in annealing temperatures, the target amplification primers cannot begin working until the operating temperature is lowered in the cycler to a point where the target amplification primers can anneal. Therefore, even when present, the target amplification primers would not be active during the first several rounds of amplification. Until the operating temperature is lowered to an appropriate annealing temperature for the target amplification primers, only the target enrichment primers would be actually generating amplicons. In certain embodiments, fresh polymerase can be added to the reaction mix before the start of amplification with target amplification primers. In some embodiments, the fresh polymerase can be a polymerase with an exonuclease activity, such as Taq polymerase. Although probes as described herein can be present from the start of the target enrichment step, in certain embodiments, probes as described herein above can be added to the reaction mix before the start of amplification with target amplification primers.

Target amplification primers can be used at high concentration for exponential amplification of the target sequences. When the Fᵢ/Rᵢ primer set is tagged, the FSP can bind the tag sequence on the Fᵢ primer and the RSP can bind the tag sequence on the Rᵢ primer. In other words, FSP/RSP recognize common primer sequences, and thus can amplify all nucleic acids that had been amplified during the target enrichment step. In one embodiment, the target amplification primers are each 10 to 50 nucleotides in length. In one embodiment, the length of the target amplification primers is from 10 to 40 nucleotides. In one embodiment, the length of the target amplification primers is from 10 to 20 nucleotides.

As used herein, a “low concentration” when used to described the concentration of the target enrichment primers means a concentration of primers that is not sufficient for exponential amplification of the given target sequence(s), but which is sufficient for target enrichment of the given target sequences. This low concentration may vary depending on the nucleotide sequence of the nucleic acid containing the target sequence to be amplified. In one embodiment, a concentration of target enrichment primers is in the range of 2 nM to less than 200 nM. In another embodiment, a concentration of target enrichment primers is in the range of 2 nM to 150 nM. In an alternate embodiment, a concentration of target enrichment primers is in the range of 2 nM to 100 nM. In yet another alternate embodiment, a concentration of target enrichment primers is in the range of 2 nM to 50 nM. Other concentration ranges outside those described above may be used if the nature of the nucleic acid sequence containing the target sequence to be amplified is such that concentrations of target enrichment primers below or above the ranges specified are required for target enrichment without exponential amplification. The various target enrichment primers may be used in different concentrations (i.e. ratios of forward to reverse primer) or at the same concentration.

As used herein, a “high concentration” when used to described the concentration of the target amplification primers (the Fᵢ/Rᵢ primers or the) means a concentration of primers that is sufficient for exponential amplification of the given target sequence. In one embodiment, a concentration of target amplification is in the range of 200 nM to 2.0 μM. In another embodiment, a concentration of target amplification primers is in the range of 200 nM to 1.0 μM. In an alternate embodiment, a concentration of target amplification primers is in the range of 200 nM to 800 nM. In yet another alternate embodiment, a concentration of target amplification primers is in the range of 200 nM to 400 nM. Other concentration ranges outside those described above may be used if the nature of the nucleic acid sequence containing the target sequence to be amplified is such that concentrations of target amplification primers below or above the ranges specified are required for exponential amplification.

As a general rule, a primer concentration in the range of 900 nM is generally used as a starting point for primer concentrations in order to achieve exponential amplification of a given target sequence. The target enrichment primers and the target amplification primers may be used in various ratios to each one another as discussed herein.

In some embodiments, more than one set of target amplification primers may be used. When more than 1 set of target amplification primers are used, the sequences of the multiple sets of target amplification primers are selected so that they are compatible with one another in the exponential amplification step. In other words, the multiple sets of amplification primers would share similar melting temperatures when binding to the binding sites on the amplified target nucleic acid and have similar amplification efficiencies. Multiple target amplification primers may be used when one or more of the detection targets are present at different titers/concentrations. In one embodiment, 2-8 sets of target amplification primers are used. In one embodiment, 2-6 sets of target amplification primers are used. In one embodiment, 2-4 sets of target amplification primers are used.

The target amplification primers can be used at high concentrations. The sequence of the target amplification primers are the same for each target sequence to be amplified if one set of target amplification primers are used, or the target amplification primers are designed to share similar amplification characteristics for each target sequence to be amplified if multiple sets of target amplification primers are used. In one embodiment, both of the target amplification primers incorporate a means for detection (e.g., a biotin tag, an enzyme label, a fluorescent tag, radionucleotide label, etc.) that enables the amplified products to be detected and/or manipulated as described below. In an alternate embodiment, only 1 of the two target amplification primers incorporates a means for detection, e.g., the Rᵢ or the RSP. In one embodiment, the means for detection may be a fluorescent element, such as, but not limited to, a CY-3® label. The fluorescent element may be directly conjugated to the super primer sequences or may be indirectly conjugated (e.g., a biotinylated primer and streptavidin-conjugated fluorophore). The detection means may be manipulated as described below.

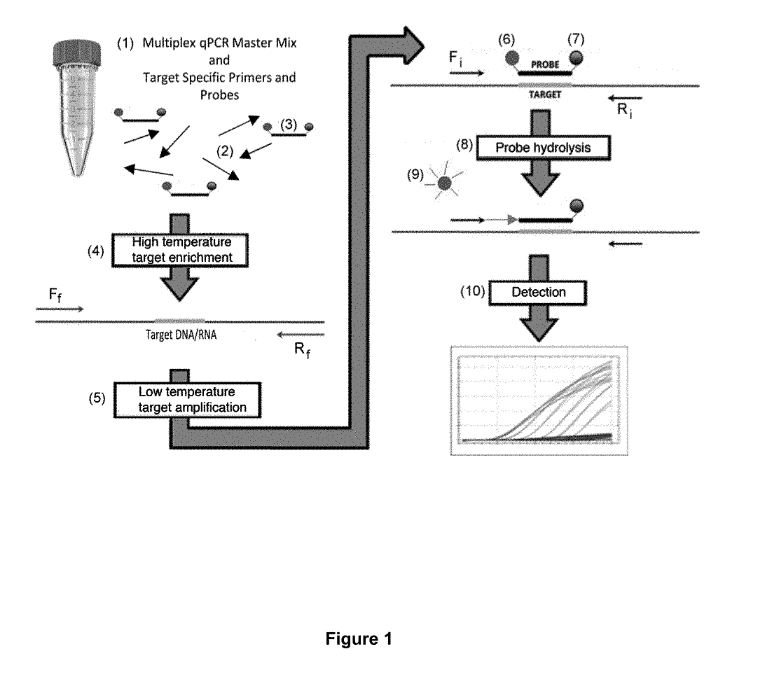

FIG. 1 illustrates an exemplary PCR method. The method begins (1) with the addition of a series of target enrichment primers (2) and probes (3) (e.g., SNP-specific probes) to a solution containing a template, along with DNA polymerase, deoxyribonucleic acid tri-phosphates (dNTPs), and various salts as necessary to create conditions appropriate for a given PCR amplification. The resulting mixture is cycled (4) through a series of high-temperature cycles in which only the F_{f} and R_{f} primers can anneal. After this (4) first amplification process, temperature in the cycler is allowed to drop (5), such that the probes and the Fᵢ and Rᵢ primers can anneal. Each probe is complementary to its respective amplification product. Each probe carries a fluorescent molecule (6) at one end and a quencher (7) at the other end that quenches the fluorescence from the fluorophore. The DNA polymerase can be a polymerase, such as Taq polymerase, with an exonuclease activity. As the polymerase moves from the target amplification primers along the templates, the polymerase will degrade the probe (8), which is bound to the template and occludes the polymerase's progress along the template. Degradation of the probe, in turn, releases the quencher and/or fluorophore from the probe, such that the fluorophore's fluorescence is no longer quenched (9), and can be detected (10) by sensors on the lid of the amplification chamber. Thus the amplification of each template can be monitored in real time (10) by following the fluorescence intensity of the corresponding probe. Because each probe can be labeled with its own color, the number of species monitored is limited only by the number of sensors available on the amplification chamber's lid. For example, in some embodiments the chamber lid can monitor at least two different fluorescent colors, for example at least 5, at least 10, at least 15, at least 20, at least 25, or at least 30 different fluorescent colors.

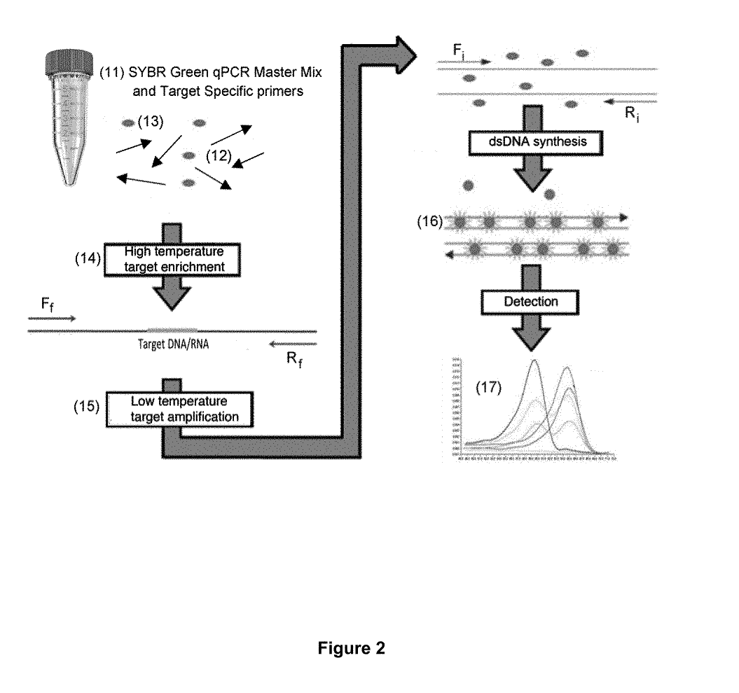

FIG. 2 illustrates another exemplary PCR method. The method begins (11) with the addition of a series of (12) target enrichment primers to a solution containing a template, along with DNA polymerase, deoxyribonucleic acid tri-phosphates (dNTPs), (13) SYBR Green® and various salts as necessary to create conditions appropriate for a given PCR amplification. Although SYBR Green® is mentioned here for illustration purposes, the same results can be obtained with many other dyes, for example by way of non-limiting illustration, in certain embodiments the fluorescent dye can be N′,N′-dimethyl-N-[4-[(E)-(3-methyl-1,3-benzothiazol-2-ylidene)methyl]-1-phenylquinolin-1-ium-2-yl]-N-propylpropane-1,3-diamine, AOAO-12, ATTO-633, ATTO-647N, or ATTO-655. The selection of such dyes is familiar to the person of ordinary skill. The resulting mixture is cycled (14) through a series of high-temperature cycles in which only the F_{f} and R_{f} primers can anneal. After this (14) first amplification process, temperature in the cycler is allowed to drop (15), such that the Fᵢ and Rᵢ primers can anneal. Because the intercalating dye activates when it binds (16) to dsDNA, the embodiment illustrated in FIG. 2 cannot be used to track different target species in real time. However, at the end of the target amplification process, the relative prevalence of each target amplicon can be assessed by a size-based assay (17), such as a melting curve. In this way, the quantitative presence of individual targets in the multiplex template mix can be assessed at the end of the target amplification process.

The ratios of the target enrichment primers (F_{f}, Fᵢ, R_{f}, and Rᵢ) used in the amplification method may be varied. Different target sequences may have different target enrichment primer requirements. Some disease agents may have DNA genomes or RNA genomes (positive or negative strand). In addition, the concentration of target amplification primers may also be varied.

Because target amplification primers are used for the exponential amplification of each target sequence, target amplification primer sequences are selected so as not to share obvious homology with any known GenBank sequences. In addition, the sequence of the target amplification primers is selected so as to share a comparable Tₘ on binding to the super primer binding sites in the amplification products to provide efficient amplification reactions. Finally, the sequence of the target amplification primers may be selected such that their priming capabilities for thermal stable DNA polymerases maybe superior to the target enrichment primers which are specific for each target sequence to be amplified.

In certain embodiments, the target sequence will be drawn from an influenza virus, for example a strain selected from the group consisting of H1N1, H1N2, H2N2, H3N2, H3N8, H4N6, H4N8, H5N1, H5N2, H5N3, H6N1, H6N2, H6N4, H7N1, H7N2, H7N3, H7N7, H7N8, H9N2, H10N5, H11N1, H11N8, and H11N9 (which includes the currently circulating avian influenza A strain, H5N1). In certain embodiments, the target sequence will be drawn from an adenovirus. In certain embodiments, the target sequence will be drawn from members of the Picornaviridae family, which includes enteroviruses and rhinoviruses. Enteroviruses also include different genera such as coxsackie viruses and echoviruses. In certain embodiments, the target sequence will be drawn from a bacterium, for example a Helicobacter species, Neisseria meningitides, Haemophilus influenzae, Escherichia coli, Listeria monocytogenes, Mycoplasma pneumoniae, Streptococcus pneumoniae, and Streptococcus agalactiae. In certain embodiments, the target sequence will be drawn from a virus selected from the group consisting of enteroviruses, coxsackievirus A, coxsackievirus B, a herpesvirus, parechovirus, and West Nile virus. In certain embodiments, the target sequence will be drawn from a genetic determinant of antibiotic resistance, such as clarithromycin resistance.

A subject of the present disclosure is also a kit comprising the components necessary for carrying out the method disclosed in all the embodiments illustrated. The kit may comprise one or more of the following: at least one set of primers to for the amplification of target sequences from a disease agent and secondary disease agent in sample from an individual suspecting of harboring the disease agent, reagents for the isolation of nucleic acid (RNA, DNA or both), reagents for the amplification of target nucleic acid from said sample (by PCR, RT-PCR or other techniques known in the art), microspheres, either with or without conjugated capturing reagents (in one embodiment, the cRTs), target sequence specific detection oligonucleotides, reagents required for positive/negative controls and the generation of first and second signals.

### FURTHER EMBODIMENTS

### Embodiment 1

A method for quantitative multiplex amplification, the method comprising:
a. amplifying a plurality of target sequences in a cycler containing a single reaction mix, wherein a pair of target enrichment primers comprising a forward flanking (F_{f}) and a reverse flanking (R_{f}) primer each hybridize to a sequence adjacent to the target sequence and wherein F_{f} and R_{f} both anneal at a first annealing temperature;
b. amplifying the plurality of target sequences in the single reaction mix, wherein a pair of target amplification primers comprising a forward inner (Fᵢ) and a reverse inner (Rᵢ) primer each hybridize to a portion of the target sequence, wherein Fᵢ and Rᵢ both anneal at a second annealing temperature, wherein the second annealing temperature is at least 3 C.° cooler than the first annealing temperature; and
wherein the single reaction mix comprises:
   i. a thermostable DNA polymerase with 5′ to 3′ exonuclease activity; and
   ii. a plurality of sequence-specific probes, wherein there is at least one probe complementary to each target sequence in the plurality, and wherein each sequence-specific probe comprises at least one fluorophore and a quencher, wherein the at least one fluorophore responds to an excitation wavelength by emitting a first fluorescence, and wherein the quencher quenches the first fluorescence prior to hydrolysis of the probe; and
wherein the cycler is equipped with filters responsive to a plurality of first fluorescence wavelengths, and wherein an illumination source periodically illuminates the single reaction mix for detection.

### Embodiment 2

The method of embodiment 1, wherein the length of each of F_{f} and R_{f} is about 10 to about 100 nucleotides.

### Embodiment 3

The method of any one of the previous embodiments, wherein the length of each of Fᵢ and Rᵢ is about 10 to about 100 nucleotides.

### Embodiment 4

The method of any one of the previous embodiments, wherein the target enrichment primers are present at about 0.002 μM to about 1.0 μM and the target amplification primers are present at about 0.1 μM to about 2.0 μM.

### Embodiment 5

The method of any one of the previous embodiments, wherein the sequence-specific probes are present at about 0.01 μM to about 0.5 μM in the single reaction mix.

### Embodiment 6

The method of any one of the previous embodiments, wherein the step a) amplification reaction includes at least two complete cycles of target enrichment and the step b) amplification reaction includes at least two complete cycles of target amplification.

### Embodiment 7

The method of embodiment 6, wherein target enrichment comprises about 10 seconds to about 1 minute at about 92° C. to about 95° C. and about 30 seconds to about 1.5 minutes at about 68° C. to about 75° C., and target amplification comprises about 10 seconds to about 1 minute at about 92° C. to about 95° C. and about 30 seconds to about 1.5 minutes at about 50° C. to about 65° C.

### Embodiment 8

The method of embodiment 7, wherein the step a) amplification reaction includes reverse transcription.

### Embodiment 9

The method of embodiment 8, wherein reverse transcription comprises about 2 to about 20 minutes at about 45° C. to about 55° C.

### Embodiment 10

The method of any one of the previous embodiments, wherein the single reaction mix comprises at least 5 distinct pairs of flanking primers.

### Embodiment 11

The method of embodiment 10, wherein the single reaction mix comprises at least 5 distinct pairs of target amplification primers.

### Embodiment 12

The method of any one of the previous embodiments, wherein at least one primer set hybridizes to a viral or a bacterial nucleotide sequence.

### Embodiment 13

The method of embodiment 12, wherein the bacteria are selected from the group consisting of Neisseria meningitidis, Haemrophilus influenzae, Escherichia coli, Listeria monocytogenes, Mycoplasma pneumoniae, Streptococcus pneumoniae, and Streptococcus agalactiae.

### Embodiment 14

The method of embodiment 12, wherein the virus is selected from the group consisting of enteroviruses, herpes viruses, parechovirus, and West Nile virus.

### Embodiment 15

The method of embodiment 14, wherein the enterovirus is selected from the group consisting of coxsackievirus A, coxsackievirus B, echovirus, and enterovirus D68.

### Embodiment 16

The method of embodiment 14, wherein the herpes viruses are selected from the group consisting of cytomegalovirus, human herpesvirus type 6, varicella zoster virus, Epstein-Barr virus, and herpes simplex viruses 1 and 2.

### Embodiment 17

The method of any one of the previous embodiments, wherein the thermostable DNA polymerase is Thermophilus aquaticus DNA polymerase.

### Embodiment 18

The method of any one of the previous embodiments, wherein the reaction comprises sequence-specific probes directed to at least 5 different sequences.

### Embodiment 19

The method of embodiment 18, wherein the reaction comprises sequence-specific probes directed to at least 10 different sequences.

### Embodiment 20

The method of embodiment 19, wherein the reaction comprises sequence-specific probes directed to at least 20 different sequences.

### Embodiment 21

The method of embodiment 20, wherein the reaction comprises sequence-specific probes directed to at least 30 different sequences.

### Embodiment 22

The method of any one of the previous embodiments, wherein the first annealing temperature is at least 5 C.° higher than the second annealing temperature.

### Embodiment 23

The method of embodiment 22, wherein the first annealing temperature is at least 10 C.° higher than the second annealing temperature.

### Embodiment 24

The method of embodiment 23, wherein the first annealing temperature is at least 20 Co higher than the second annealing temperature.

### Embodiment 25

The method of any one of the previous embodiments, wherein the quantitative amplification is a real-time polymerase chain reaction amplification.

### Embodiment 26

A method for quantitative multiplex amplification, the method comprising:
a. amplifying a plurality of target sequences in a cycler containing a single reaction mix, wherein a pair of target enrichment primers comprising a forward flanking (F_{f}) and a reverse flanking (R_{f}) primer each hybridize to a sequence adjacent to the target sequence and wherein F_{f} and R_{f} both anneal at a first annealing temperature;
b. amplifying the plurality of target sequences in the single reaction mix, wherein a pair of target amplification primers comprising a forward inner (Fᵢ) and a reverse inner (Rᵢ) primer each hybridize to a portion of the target sequence, wherein Fᵢ and Rᵢ both anneal at a second annealing temperature, wherein the second annealing temperature is at least 3 C.° cooler than the first annealing temperature; and
wherein the single reaction mix comprises:
   i. a fluorescent dye that emits a more intense fluorescence at a given wavelength when bound to double-stranded DNA (dsDNA) than when not bound to dsDNA;
wherein the cycler is equipped with filters responsive to a single fluorescence wavelength, and wherein an illumination source periodically illuminates the reaction mix for detection.

### Embodiment 27

The method of embodiment 26, wherein the length of each of F_{f} and R_{f} is about 10 to about 100 nucleotides.

### Embodiment 28

The method of any one of the previous embodiments, wherein the length of each of Fᵢ and Rᵢ is about 10 to about 100 nucleotides.

### Embodiment 29

The method of any one of the previous embodiments, wherein the target enrichment primers are present at about 0.002 μM to about 1.0 μM and the target amplification primers are present at about 0.1 μM to about 1.0 μM.

### Embodiment 30

The method of any one of the previous embodiments, wherein the step a) amplification reaction includes at least 15 complete cycles of target enrichment and the step b) amplification reaction includes at least 15 complete cycles of target amplification.

### Embodiment 31

The method of any one of the previous embodiments, further comprising c) gradually melting the amplicons by raising temperature in the cycler no faster than about 0.5 Co per second.

### Embodiment 32

The method of embodiment 31, comprising measuring fluorescence at each 0.5 C.° increase.

### Embodiment 33

The method of embodiment 31, wherein target enrichment comprises about 10 seconds to about 1 minute at about 92° C. to about 95° C. and about 30 seconds to about 1.5 minutes at about 68° C. to about 75° C., and target amplification comprises about 10 seconds to about 1 minute at about 92° C. to about 95° C. and about 30 seconds to about 1.5 minutes at about 50° C. to about 65° C.

### Embodiment 34

The method of embodiment 33, wherein the step a) amplification reaction includes reverse transcription.

### Embodiment 35

The method of embodiment 34, wherein reverse transcription comprises about 10 to about 20 minutes at about 45° C. to about 55° C.

### Embodiment 36

The method of any one of the previous embodiments, wherein the single reaction mix comprises at least 3 distinct pairs of flanking primers.

### Embodiment 37

The method of any one of the previous embodiments, wherein the single reaction mix comprises 3 or more pairs of target amplification primers.

### Embodiment 38

The method of any one of the previous embodiments, wherein at least one primer set hybridizes to a viral or a bacterial nucleotide sequence.

### Embodiment 39

The method of embodiment 38, wherein the bacteria are selected from the group consisting of Helicobacter species, Neisseria meningitides, Haemophilus influenzae, Escherichia coli, Listeria monocytogenes, Mycoplasma pneumoniae, Streptococcus pneumoniae, and Streptococcus agalactiae.

### Embodiment 40

The method of embodiment 38, wherein the virus is selected from the group consisting of enteroviruses, herpes viruses, and West Nile virus.

### Embodiment 41

The method of embodiment 40, wherein the enterovirus is selected from the group consisting of coxsackievirus A, coxsackievirus B, echovirus, and enterovirus D68.

### Embodiment 42

The method of embodiment 40, wherein the herpes viruses are selected from the group consisting of cytomegalovirus, human herpesvirus type 6, varicella zoster virus, Epstein-Barr virus, and herpes simplex viruses 1 and 2.

### Embodiment 43

The method of any one of the previous embodiments, wherein the thermostable DNA polymerase is Thermophilus aquaticus DNA polymerase.

### Embodiment 44

The method of any one of the previous embodiments, wherein the first annealing temperature is at least 5 C.° higher than the second annealing temperature.

### Embodiment 45

The method of embodiment 44, wherein the first annealing temperature is at least 10 C.° higher than the second annealing temperature.

### Embodiment 46

The method of embodiment 45, wherein the first annealing temperature is at least 20 Co higher than the second annealing temperature.

### Embodiment 47

The method of any one of the previous embodiments, wherein:
(a) the fluorescent dye is N′,N′-dimethyl-N-[4-[(E)-(3-methyl-1,3-benzothiazol-2-ylidene)methyl]-1-phenylquinolin-1-ium-2-yl]-N-propylpropane-1,3-diamine and the given wavelength is 520 nm; or
(b) the fluorescent dye is AOAO-12 and the given wavelength is 530 nm; or
(c) the fluorescent dye is ATTO-633 and the given wavelength is 657 nm; or
(d) the fluorescent dye is ATTO-647N and the given wavelength is 669 nm; or
(e) the fluorescent dye is ATTO-655 and the given wavelength is 684 nm.

### EXAMPLES

### Example 1. Preamplification Functionality and Probe Stability

To test whether the probe can be added to a multiplex PCR reaction at the start of the process, without being hydrolyzed during a first round of high-temperature amplification, reactions were assembled with primers directed to the target sequences shown in Table 1. Individual reaction tubes each contained probes and primers directed to their respective targets. A control plate was assembled with water in place of the target enrichment primers to give a baseline CT for comparison. Each reaction for a DNA virus shown in Table 1 below (except HHV6) was 10 μL in volume, with 5 μL of TaqMan® Gene Expression Master Mix (THERMO FISHER), 3.5 μL template, 0.5 μL of Fᵢ/Rᵢ/probe mix, and 1 μL of F_{f}/R_{f} mix (or water, in the control, instead of the combined 1.5 μL of primers and probes). Each reaction for an RNA virus shown in Table 1 below (except EV) was 10 μL in volume, with 5 μL of RNA-to-CT 1-Step® kit Master Mix (THERMO FISHER), 3.5 μL template, 0.5 μL of Fᵢ/Rᵢ/probe mix, and 1 μL of F_{f}/R_{f} mix (or water, in the control, instead of the combined 1.5 μL of primers and probes). The HHV6 reactions each contained 2.5 μL of 4× TaqPath 1-Step Multiplex Master Mix (THERMO FISHER), 1.5 μL probe/primer mix, 2.9 μL water, and 3.1 μL template. The HHV6 reaction did not use Fᵢ/Rᵢ primers. Primer concentrations are shown in Table 1 below. No-template controls were also run. The amplifications were run in a CFX96 qPCR System (BIO RAD). DNA viruses were amplified according to the following program: UDG incubation at 50° C. for 2 min; Enzyme activation at 95° C. for 10 min; Preamplification through 15 cycles of 95° C. for 15 see, 72° C. for 1 min; Amplification through 40 cycles of 95° C. for 15 sec and 60° C. for 1 min. RNA viruses were amplified according to the following program: Reverse transcription at 50° C. for 15 min; Enzyme activation at 95° C. for 10 min; Preamplification through 15 cycles of 95° C. for 15 sec, 72° C. for 1 min; Amplification through 40 cycles of 95° C. for 15 sec and 60° C. for 1 min. All reactions included a probe specific for the target sequence with FAM as the dye and MGB as the quencher. Two different F_{f} primers were used simultaneously in the amplification of EV 5′UTR and HHV6 U83.

TABLE 1

| | Gene | | Melt Temp. | Rxn. Conc. |
|---|---|---|---|---|
| Virus Target | Target | Oligos | (° C.) | (nM) |
| Cytomegalovirus | UL75 | F_{f} | 75.3 | 400 |
| (CMV) | | R_{f} | 75.5 | 400 |
| | | Fᵢ | 59 | 900 |
| | | Rᵢ | 58 | 900 |
| | | Probe | 69 | 250 |
| Epstein-barr | BGLF4 | F_{f} | 75.5 | 400 |
| virus (EBV) | | R_{f} | 75.4 | 400 |
| | | Fᵢ | 58 | 900 |
| | | Rᵢ | 60 | 900 |
| | | Probe | 69 | 250 |
| Human herpes 6 | U83 | 1-F_{f} | 75 | 750 |
| virus (HHV6) | | 2-F_{f} | 74.5 | 750 |
| | | R_{f} | 72.6 | 1300 |
| | | Probe | 68 | 250 |
| Herpes simplex | US4 | F_{f} | 74.1 | 400 |
| virus 1 (HSV1) | | R_{f} | 75.6 | 400 |
| | | Fᵢ | 59 | 900 |
| | | Rᵢ | 58 | 900 |
| | | Probe | 70 | 250 |
| Herpes simplex | US4 | F_{f} | 74 | 400 |
| virus 2 (HSV2) | | R_{f} | 75.8 | 400 |
| | | Fᵢ | 58 | 900 |
| | | Rᵢ | 58 | 900 |
| | | Probe | 69 | 250 |
| Varicella zoster | IE62 | F_{f} | 74.3 | 400 |
| virus (VZV) | | R_{f} | 74.3 | 400 |
| | | Fᵢ | 58 | 900 |
| | | Rᵢ | 59 | 900 |
| | | Probe | 68 | 250 |
| Enterovirus (EV) | 5′UTR | 1-F_{f} | 74.8 | 750 |
| | | 2-F_{f} | 73.6 | 750 |
| | | R_{f} | 73.6 | 1300 |
| | | Probe | 70 | 250 |
| Enterovirus D68 | VP1 | F_{f} | 74.2 | 400 |
| (EVD68) | | R_{f} | 72.3 | 400 |
| | | Fᵢ | 56.6 | 900 |
| | | Rᵢ | 59.4 | 900 |
| | | Probe | 68 | 250 |
| Human parechovirus | 5′UTR | F_{f} | 74 | 400 |
| (HPeV) | | R_{f} | 73.4 | 400 |
| | | Fᵢ | 59 | 900 |
| | | Rᵢ | 60 | 900 |
| | | Probe | 69 | 250 |
| West Nile | 3′UTR | F_{f} | 74 | 400 |
| virus (WNV) | | R_{f} | 74.7 | 400 |
| | | Fᵢ | 61 | 900 |
| | | Rᵢ | 54 | 900 |
| | | Probe | 73 | 250 |

Each reaction was run in triplicate. To further test whether the probes can function in a two-step enrichment-then-amplification process, each set of targets was tested both with and without the enrichment step. Table 2 shows the results as average Ct and standard deviation values from the three reactions. As can be seen, each probe was active by the time of data collection, even after an enrichment-then-amplification process. This demonstrates that the process does not hydrolyze the probe before useful results can be collected. The control reactions with primers but without template showed no amplification in any condition.

TABLE 2

| | [Virus] | No enrichment step | | Enrichment step | |
|---|---|---|---|---|---|
| Virus | cfu/mL or | Avg. | Std. | Avg. | Std. |
| Target | copies/mL | Ct | Dev. | Ct | Dev. |
| CMV | 1000 | 22.42 | 0.03 | 8.23 | 0.05 |
| (pfu/mL) | 100 | 25.89 | 0.17 | 11.57 | 0.01 |
| | 10 | 29.38 | 0.12 | 15.17 | 0.18 |
| | 1 | 32.76 | 0.49 | 18.59 | 0.34 |
| | None | — | — | — | — |
| EBV | 215000 | 26.55 | 0.07 | 11.73 | 0.02 |
| (cp/mL) | 21500 | 29.76 | 0.07 | 15.04 | 0.07 |
| | 2150 | 33.58 | 0.93 | 18.26 | 0.23 |
| | 215 | 38.00 | 1.25 | 21.40 | 0.13 |
| | None | — | — | — | — |
| HHV6 | 1750 | | | 14.29 | 0.13 |
| (cp/mL) | 175 | | | 17.11 | 0.46 |
| | 17.5 | | | 19.94 | 0.55 |
| | 1.75 | | | — | — |
| | None | | | — | — |
| HSV1 | 1 million | 23.74 | 0.11 | 9.31 | 0.10 |
| (pfu/mL) | 100000 | 27.09 | 0.05 | 12.82 | 0.05 |
| | 10000 | 30.45 | 0.17 | 16.19 | 0.23 |
| | 1000 | 34.69 | 0.20 | 19.43 | 0.42 |
| | 100 | 36.49 | 0.15 | 22.49 | 0.52 |
| | None | — | — | — | — |
| HSV2 | 1000 | 23.78 | 0.10 | 10.79 | 0.09 |
| (pfu/mL) | 100 | 27.18 | 0.04 | 14.23 | 0.05 |
| | 10 | 30.64 | 0.07 | 17.88 | 0.20 |
| | 1 | 34.35 | 1.03 | 28.39 | 8.53 |
| | None | — | — | — | — |
| VZV | 100 | 22.80 | 0.11 | 8.26 | 0.16 |
| (pfu/mL) | 10 | 26.06 | 0.08 | 11.84 | 0.04 |
| | 1 | 29.34 | 0.20 | 15.17 | 0.11 |
| | None | — | — | — | — |
| EVD68 | 100000 | 24.95 | 0.34 | 16.47 | 0.35 |
| (pfu/mL) | None | — | — | — | — |
| HPeV | 100000 | 25.55 | 0.06 | 22.32 | 0.12 |
| (pfu/mL) | 10000 | 28.99 | 0.13 | 23.87 | 0.12 |
| | 1000 | 32.30 | 0.16 | 27.05 | 0.15 |
| | 100 | 36.78 | 0.77 | 29.96 | 0.31 |
| | None | — | — | — | — |
| WNV | 100000 | 13.50 | 0.08 | 7.15 | 0.14 |
| (pfu/mL) | 10000 | 17.24 | 0.04 | 6.00 | 0.08 |
| | 1000 | 20.66 | 0.05 | 8.54 | 0.24 |
| | 100 | 24.12 | 0.06 | 11.85 | 0.05 |
| | None | — | — | — | — |

### Example 2. Real-Time Multiplex Quantification of Viruses

To confirm that multiple probes and primer sets would be able to function together in a single reaction, three mixes of viral templates and three corresponding mixtures of primers and probes were assembled and tested. All template mixtures were assembled in a final volume of 100 μL. Their compositions are shown in Table 3. A plasmid containing the ascorbate peroxidase gene (APX1) of Arabidopsis thaliana was included in each panel as an internal control.

TABLE 3

| Panel 1 | | | Panel 2 | | | Panel 3 | | |
|---|---|---|---|---|---|---|---|---|
| Template | Per uL | | Template | Per LiL | | Template | Per uL | |
| EV | 1000 | pfu | HSV1 | 100000 | pfu | CMV | 10000 | pfu |
| EVD68 | 1000 | pfu | HSV2 | 100 | pfu | HHV6 | 223 | copies |
| HPeV 1 | 1000 | pfu | EBV | 215 | copies | VZV | 100 | pfu |
| WNV 1 | 1000 | pfu | APX1 | 1 million copies | | APX1 | 1 million copies | |
| APX1 | 5000 | copies | | | | | | |

Probes

| Target | Dye/Quencher | Target | Dye/Quencher | Target | Dye/Quencher |
|---|---|---|---|---|---|
| EV | FAM/MGB | HSV1 | VIC/MGB | CMV | FAM/MGB |
| EVD68 | VIC/MGB | HSV2 | NED/MGB | HHV6B | ABY/QSY |
| HPeV 1 | JUN/QSY | EBV | FAM/MGB | VZV | VIC/MGB |
| WNV 1 | NED/MGB | APX1 | Cy5/IB-RQ | APX1 | Cy5/IB-RQ |
| APX1 | Cy5/IB-RQ | | | | |

Flanking primers, amplification primers (i.e., Fᵢ/Rᵢ primers) and probes were designed against each of the template targets in Table 3. One of ordinary skill in the art is well aware on how to make primers and probes for template targets. Three sets of primers and probes were assembled, one for each of the template panels in Table 3. The dye/quencher combinations for each probe are also shown in Table 3. Reactions were assembled in the same manner and at the same concentrations as in Example 1. Each primer set was tested on both the mixed template panels shown in Table 3 and on individual samples of single templates to test the selectivity of the primer/probe mixes. Amplification reactions were run in the same sequence as in Example 1.

Table 4 shows the results of the multiplex amplification of panel 1. As in Table 2 above, the Ct values shown represent the average of a triplicate measurement. As can be seen, all templates except EV were each selectively amplified and selectively detected in the multiplex. The EV primers and probe show inclusivity to EVD68 template, so a positive multiplex result for EV may require additional confirmation to be truly diagnostic. As expected, the no-template control showed no amplification.

TABLE 4

| Template | Dye | Avg. Ct | Std. Dev. | Dye | Avg. Ct | Std. Dev. |
|---|---|---|---|---|---|---|
| APX1 | CY5 | 14.07 | 0.09 | JUN | — | — |
| EV | CY5 | — | — | JUN | — | — |
| EVD68 | CY5 | — | — | JUN | — | — |
| HPeV 1 | CY5 | — | — | JUN | 19.85 | 0.26 |
| WNV 1 | CY5 | — | — | JUN | — | — |
| Multiplex | CY5 | 13.35 | 0.10 | JUN | 20.19 | 0.29 |
| No template | CY5 | — | — | JUN | — | — |
| APX1 | FAM | — | — | NED | — | — |
| EV | FAM | 14.73 | 0.10 | NED | — | — |
| EVD68 | FAM | 27.15 | — | NED | — | — |
| HPeV 1 | FAM | — | — | NED | — | — |
| WNV 1 | FAM | — | — | NED | 15.63 | 0.11 |
| Multiplex | FAM | 14.60 | 0.27 | NED | 14.92 | 0.25 |
| No template | FAM | — | — | NED | — | — |
| APX1 | VIC | — | — | | | |
| EV | VIC | — | — | | | |
| EVD68 | VIC | 13.46 | 0.06 | | | |
| HPeV 1 | VIC | — | — | | | |
| WNV 1 | VIC | — | — | | | |
| Multiplex | VIC | 13.28 | 0.16 | | | |
| No template | VIC | — | — | | | |

Table 5 shows the results of the multiplex amplification of panel 2. As can be seen, each target was selectively amplified and selectively detected in the multiplex. As expected, the no-template control showed no amplification.

TABLE 5

| Template | Dye | Avg. Ct | Std. Dev. | Dye | Avg. Ct | Std. Dev. |
|---|---|---|---|---|---|---|
| APX1 | CY5 | 7.52 | 0.12 | VIC | — | — |
| EBV | CY5 | — | — | VIC | — | — |
| HSV1 | CY5 | — | — | VIC | 13.04 | 0.17 |
| HSV2 | CY5 | — | — | VIC | — | — |
| Multiplex | CY5 | 7.59 | 0.04 | VIC | 13.49 | 0.01 |
| No template | CY5 | — | — | VIC | — | — |
| APX1 | FAM | — | — | NED | — | — |
| EBV | FAM | 12.29 | 0.00 | NED | — | — |
| HSV1 | FAM | — | — | NED | — | — |
| HSV2 | FAM | — | — | NED | 17.31 | 0.14 |
| Multiplex | FAM | 11.71 | 0.06 | NED | 16.33 | 0.11 |
| No template | FAM | — | — | NED | — | — |

Table 6 shows the results of the multiplex amplification of panel 3. As can be seen, each target was selectively amplified and selectively detected in the multiplex. As expected, the no-template control showed no amplification.

TABLE 6

| Template | Dye | Avg. Ct | Std. Dev. | Dye | Avg. Ct | Std. Dev. |
|---|---|---|---|---|---|---|
| APX1 | CY5 | 7.38 | 0.12 | FAM | — | — |
| CMV | CY5 | — | — | FAM | — | — |
| HHV6B | CY5 | — | — | FAM | 11.27 | 0.21 |
| VZV | CY5 | — | — | FAM | — | — |
| Multiplex | CY5 | 7.11 | 0.04 | FAM | 10.25 | 0.08 |
| No template | CY5 | — | — | FAM | — | — |
| APX1 | NED | — | — | VIC | — | — |
| CMV | NED | 8.01 | 0.36 | VIC | — | — |
| HHV6B | NED | — | — | VIC | — | — |
| VZV | NED | — | — | VIC | 11.31 | 0.14 |
| Multiplex | NED | 7.61 | 0.04 | VIC | 11.58 | 0.09 |
| No template | NED | — | — | VIC | — | — |

### Example 3. Real-Time Multiplex Quantification of Bacteria

As in Example 2 above, similar tests were run with two different panels of bacteria to assess the ability of individual primer pairs to amplify a given bacterial target sequence selectively when mixed with other, off-target primer pairs. All template mixtures were assembled in a final volume of 100 μL. Their compositions are shown in Table 7. The APX1 plasmid was included in each panel as an internal control.

TABLE 7

| Panel 15 | | Panel 14 | |
|---|---|---|---|
| Template | Per μL | Template | Per μL |
| H. influenzæ | 1000 cfu | E. coli | 1000 cfu |
| M. pneumoniæ | 1000 cfu | S. agalactiæ | 1000 cfu |
| N. meningitidis | 1000 cfu | L. monocytogenes | 10000 cfu |
| S. pneumoniæ | 1000 cfu | APX1 | 5000 copies |
| APX1 | 5000 copies | | |

Probes

| Target | Dye/Quencher | Target | Dye/Quencher |
|---|---|---|---|
| H. influenzæ | ABY/QSY | E. coli | ABY/QSY |
| M. pneumoniæ | VIC/QSY | S. agalactiæ | VIC/MGB |
| N. meningitidis | JUN/QSY | L. monocytogenes | FAM/QSY |
| S. pneumoniæ | FAM/MGB | APX1 | Cy5/IB-RQ |
| APX1 | Cy5/IB-RQ | | |

Reactions were assembled in the same manner and at the same concentrations as in Examples 1 & 2. Each primer set was tested on both the mixed template panels shown in Table 7 and on individual samples of single templates to test the selectivity of the primer/probe mixes. Amplification reactions were run in the same sequence as in Examples 1 & 2. Table 8 shows the results of the multiplex amplification of panel 5. As above, the Ct values shown represent the average of a triplicate measurement. As can be seen, all templates were each selectively amplified and selectively detected in the multiplex. As expected, the no-template control showed no amplification.

TABLE 8

| | Dye | | Std. | Dye | | |
|---|---|---|---|---|---|---|
| Template | channel | Avg. Ct | Dev. | channel | Avg. Ct | Std. Dev. |
| APX1 | CY5 | 15.02 | 0.01 | JUN | — | — |
| H. influenzce | CY5 | — | — | JUN | — | — |
| M. pneumonice | CY5 | — | — | JUN | — | — |
| N. meningitidis | CY5 | — | — | JUN | 15.53 | 0.03 |
| S. pneumonice | CY5 | — | — | JUN | — | — |
| Multiplex | CY5 | 14.55 | 0.16 | JUN | 15.26 | 0.14 |
| No template | CY5 | — | — | JUN | — | — |
| APX1 | FAM | — | — | ABY | — | — |
| H. influenzce | FAM | — | — | ABY | 17.50 | 0.31 |
| M. pneumonice | FAM | — | — | ABY | — | — |
| N. meningitidis | FAM | — | — | ABY | — | — |
| S. pneumonice | FAM | 20.05 | 0.38 | ABY | — | — |
| Multiplex | FAM | 19.15 | 0.40 | ABY | 16.26 | 0.09 |
| No template | FAM | — | — | ABY | — | — |
| APX1 | VIC | — | — | | | |
| H. influenzce | VIC | — | — | | | |
| M. pneumonice | VIC | 12.58 | 0.09 | | | |
| N. meningitidis | VIC | — | — | | | |
| S. pneumonice | VIC | — | — | | | |
| Multiplex | VIC | 12.55 | 0.05 | | | |
| No template | VIC | — | — | | | |

Table 9 shows the results of the multiplex amplification of panel 4. As can be seen, each target was selectively amplified and selectively detected in the multiplex. As expected, the no-template control showed no amplification.

TABLE 9

| | Dye | | | Dye | Avg. | Std. |
|---|---|---|---|---|---|---|
| Template | channel | Avg. Ct | Std. Dev. | channel | Ct | Dev. |
| APX1 | CY5 | 15.07 | 0.22 | FAM | — | — |
| E. coli | CY5 | — | — | FAM | — | — |
| S. agalactice | CY5 | — | — | FAM | — | — |
| L. monocytogenes | CY5 | — | — | FAM | 22.14 | 0.38 |
| Multiplex | CY5 | 14.90 | 0.11 | FAM | 22.00 | 0.83 |
| No template | CY5 | — | — | FAM | — | — |
| APX1 | ABY | — | — | VIC | — | — |
| E. coli | ABY | 15.67 | 0.15 | VIC | — | — |
| S. agalactice | ABY | — | — | VIC | 19.95 | 0.41 |
| L. monocytogenes | ABY | — | — | VIC | — | — |
| Multiplex | ABY | 15.39 | 0.20 | VIC | 18.34 | 0.23 |
| No template | ABY | — | — | VIC | — | — |

These data demonstrate that the methods described herein provide a quick and accurate way to quantify the presence of multiple nucleic acid target sequences in a single sample.

The above examples are merely illustrative, and do not limit this disclosure in any way. All patents and patent applications cited herein are incorporated by reference to the extent allowed. The references discussed herein are provided solely for their disclosure prior to the filing date of the present application. Nothing herein is to be construed as an admission that the inventors are not entitled to antedate such disclosure by virtue of prior disclosure.

What is claimed is:

1. A method for quantitative multiplex amplification, the method comprising:
a) amplifying a plurality of target sequences in a cycler containing a single reaction mix, wherein a pair of target enrichment primers comprising a forward flanking (F_{f}) and a reverse flanking (R_{f}) primer each hybridize to a sequence adjacent to the target sequence and wherein F_{f} and R_{f} both anneal at a first annealing temperature;
b) amplifying the plurality of target sequences in the single reaction mix, wherein a pair of target amplification primers comprising a forward inner (Fᵢ) and a reverse inner (Rᵢ) primer each hybridize to a portion of the target sequence, wherein Fᵢ and Rᵢ both anneal at a second annealing temperature, wherein the second annealing temperature is at least 3 C.° cooler than the first annealing temperature; and
wherein the single reaction mix comprises:
i) a thermostable DNA polymerase with 5′ to 3′ exonuclease activity; and
ii) a plurality of sequence-specific probes, wherein there is at least one probe complementary to each target sequence in the plurality, and wherein each sequence-specific probe comprises at least one fluorophore and a quencher, wherein the at least one fluorophore responds to an excitation wavelength by emitting a first fluorescence, and wherein the quencher quenches the first fluorescence prior to hydrolysis of the probe; and
wherein the cycler is equipped with filters responsive to a plurality of first fluorescence wavelengths, and wherein an illumination source periodically illuminates the single reaction mix for detection.

2. The method of claim 1, wherein the length of each of F_{f} and R_{f} is about 10 to about 100 nucleotides.

3. The method of claim 1, wherein the length of each of Fᵢ and Rᵢ is about 10 to about 100 nucleotides.

4. The method of claim 1, wherein the target enrichment primers are present at about 0.002 μM to about 1.0 μM and the target amplification primers are present at about 0.1 μM to about 2.0 μM.

5. The method of claim 1, wherein the sequence-specific probes are present at about 0.01 μM to about 0.5 μM in the single reaction mix.

6. The method of claim 1, wherein the step a) amplification reaction includes at least two complete cycles of target enrichment and the step b) amplification reaction includes at least two complete cycles of target amplification.

7. The method of claim 6, wherein target enrichment comprises
about 10 seconds to about 1 minute at about 92° C. to about 95° C. and
about 30 seconds to about 1.5 minutes at about 68° C. to about 75° C., and target amplification comprises
about 10 seconds to about 1 minute at about 92° C. to about 95° C. and
about 30 seconds to about 1.5 minutes at about 50° C. to about 65° C.

8. The method of claim 7, wherein the step a) amplification reaction includes reverse transcription.

9. The method of claim 8, wherein reverse transcription comprises
about 2 to about 20 minutes at about 45° C. to about 55° C.

10. The method of claim 1, wherein the single reaction mix comprises at least 5 distinct pairs of flanking primers.

11. The method of claim 10, wherein the single reaction mix comprises at least 5 distinct pairs of target amplification primers.

12. The method of claim 1, wherein at least one primer set hybridizes to a viral or a bacterial nucleotide sequence.

13. The method of claim 12, wherein the bacteria are selected from the group consisting of Neisseria meningitidis, Haemophilus influenzae, Escherichia coli, Listeria monocytogenes, Mycoplasma pneumoniae, Streptococcus pneumoniae, and Streptococcus agalactiae.

14. The method of claim 12, wherein the virus is selected from the group consisting of enteroviruses, herpes viruses, parechovirus, and West Nile virus.

15. The method of claim 14, wherein the enterovirus is selected from the group consisting of coxsackievirus A, coxsackievirus B, echovirus, and enterovirus D68.

16. The method of claim 14, wherein the herpes viruses are selected from the group consisting of cytomegalovirus, human herpesvirus type 6, varicella zoster virus, Epstein-Barr virus, and herpes simplex viruses 1 and 2.

17. The method of claim 1, wherein the thermostable DNA polymerase is Thermophilus aquaticus DNA polymerase.

18. The method of claim 1, wherein the reaction comprises sequence-specific probes directed to at least 5 different sequences.

19. The method of claim 1, wherein the first annealing temperature is at least 5 C.° higher than the second annealing temperature.

20. A method for quantitative multiplex amplification, the method comprising:
a) amplifying a plurality of target sequences in a cycler containing a single reaction mix, wherein a pair of target enrichment primers comprising a forward flanking (F_{f}) and a reverse flanking (R_{f}) primer each hybridize to a sequence adjacent to the target sequence and wherein F_{f} and R_{f} both anneal at a first annealing temperature;
b) amplifying the plurality of target sequences in the single reaction mix, wherein a pair of target amplification primers comprising a forward inner (Fᵢ) and a reverse inner (Rᵢ) primer each hybridize to a portion of the target sequence, wherein Fᵢ and Rᵢ both anneal at a second annealing temperature, wherein the second annealing temperature is at least 3 C.° cooler than the first annealing temperature; and
wherein the single reaction mix comprises a fluorescent dye that emits a more intense fluorescence at a given wavelength when bound to double-stranded DNA (dsDNA) than when not bound to dsDNA; and
wherein the cycler is equipped with filters responsive to a single fluorescence wavelength; and
wherein an illumination source periodically illuminates the reaction mix for detection.
